(19) United States
(12) Patent Application Publication
(10) Pub. No.: US 2022/0148716 A1
(43) Pub. Date: May 12, 2022

(54) MONITORING SYSTEM RESPONSIVE TO MULTI-FUNCTIONAL VALIDATION

(71) Applicant: Allis Technology Pty Ltd, Illawong (AU)

(72) Inventors: Marrianne McGhee, Illawong (AU); Natalie Palakawong, Bangkok (TH); Kartheek Munigoti Shankar Rao, Melbourne (AU)

(21) Appl. No.: 17/580,983

(22) Filed: Jan. 21, 2022

Related U.S. Application Data
(63) Continuation of application No. 16/397,989, filed on Apr. 29, 2019, now abandoned.

(30) Foreign Application Priority Data
Apr. 30, 2018 (AU) 2018901422

(51) Int. Cl. G16H 40/20; G16H 40/63

(52) U.S. Cl. CPC G16H 40/20; G16H 40/63

(57) ABSTRACT

A perioperative monitoring system which includes a selection module configured to receive operational requirements, a database of conditions, a condition module responsive to the selection module and in communication with the database of conditions. The condition module is configured to select conditions from the database based on the received operational requirements. At least one validation system receives the selected conditions from the condition module and determines a validation. The one validation system includes a filter that filters selected conditions based on a functional selection in order to provide a functional configuration.

### CROSS REFERENCE TO RELATED APPLICATIONS

This application is a continuation of U.S. patent application Ser. No. 16/397,989, titled “MONITORING SYSTEM RESPONSIVE TO MULTI-FUNCTIONAL VALIDATION,” filed Apr. 29, 2019, which claims priority to Australian Patent Application No. 2018901422, filed Apr. 30, 2018, the entire contents of each of which are incorporated by reference herein.

### TECHNICAL FIELD

The present disclosure relates, generally, to a monitoring system for the management of operating room resources and, more particularly, to a perioperative monitoring system responsive to multi-functional and multi-stage validation.

### BACKGROUND

In the operation of complex systems where several functions interoperate there is room for error not only within each functional role, but in particular where the functions interface, require handover, and responsibilities and roles are not clearly demarcated. Such complex systems may be, for example, multi-location security systems, fleet management, hospital operating rooms, etc.

The smooth and safe operation of complex systems may be improved by using automated or semi-automated systems, but automating such operations becomes more difficult the more complex the system is. This is particularly the case where multiple functions within the overall operation need to interface with one another, so that the functional roles must be clearly demarcated and managed individually, as well as include defined interfaces with related functions.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

As used herein, the term “validation” refers to the assessment of an action, decision, plan, or transaction to establish that it is (1) correct, (2) complete, (3) being implemented (and/or recorded) as intended, and (4) delivering the intended outcome.

In one aspect there is provided a perioperative monitoring system which includes a selection module configured to receive operational requirements; a database of conditions; a condition module responsive to the selection module and in communication with the database of conditions, wherein the condition module is configured to select conditions from the database based on the received operational requirements; and at least one validation system that receives the selected conditions from the condition module and determines a validation, wherein the at least one validation system includes a filter that filters selected conditions based on a functional selection in order to provide a functional configuration.

The monitoring system may further include a lock module that locks or unlocks a context based on the validation. The validation system may further include two or more validation systems each associated with a respective functional configuration, and the system may determine successful system validation based on a validation of at least two of the functional configurations. The two or more respective functional configurations may complement one another so as to be interoperable.

The conditions may include procedure parameters associated with a selected procedure or procedure category.

The database of conditions may include one or more checklists selected from the group consisting of: a Preparation Specification, an Operating Room Specification, a Patient Positioning Specification and an Equipment Specification. The operational requirements may include a procedure category and the one or more checklists may be selected based on the procedure category. The one or more checklists may be user-configurable.

The monitoring system may further include a computing system or device having a display; a user interface provided on the display, the user interface functionally configurable based on at least one of: the computing system or device type, the computing system or device location, and the functional selection.

In another aspect there is provided a perioperative multi-stage validation system which includes a function unit configured to provide a functional selection; a filter that filters selected conditions in a database of conditions based on the functional selection in order to provide a functional configuration; a sensor input that provides at least one parameter for validation; and a validator that validates the at least one parameter against the functional configuration to provide a validation.

The selected conditions may be selected from the database of conditions based on at least one operational requirement.

The at least one operational requirement may include at least one of: a procedure identifier, an item identifier, and a context identifier. The procedure identifier may be received from the database. The procedure identifier may be received from a user input. The procedure identifier may be configurable.

The functional configuration may include one or more checklists selected from the group consisting of: a Preparation Specification, an Operating Room Specification, a Patient Positioning Specification and an Equipment Specification.

The multi-stage validation system may be associated with another interoperable perioperative multi-stage validation system and may be in communication with the other perioperative multi-stage validation system in order to provide the validation.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the disclosure are now described by way of example with reference to the accompanying drawings in which:

FIG. 1 is a schematic representation of an embodiment of a monitoring system;

FIG. 2 is a flow diagram illustrating a method of monitoring;

FIG. 3 is a schematic representation of an embodiment of a multi-stage validation system of the system of FIG. 1;

FIG. 4 is a flow diagram illustrating a method of validating;

FIG. 5 is a schematic representation of an exemplary embodiment of a monitoring system; and

FIG. 6 is a schematic representation of an exemplary implementation of the multi-functional monitoring system of FIG. 1.

In the drawings, like reference numerals designate similar parts.

### DESCRIPTION OF EMBODIMENTS

Monitoring System 100

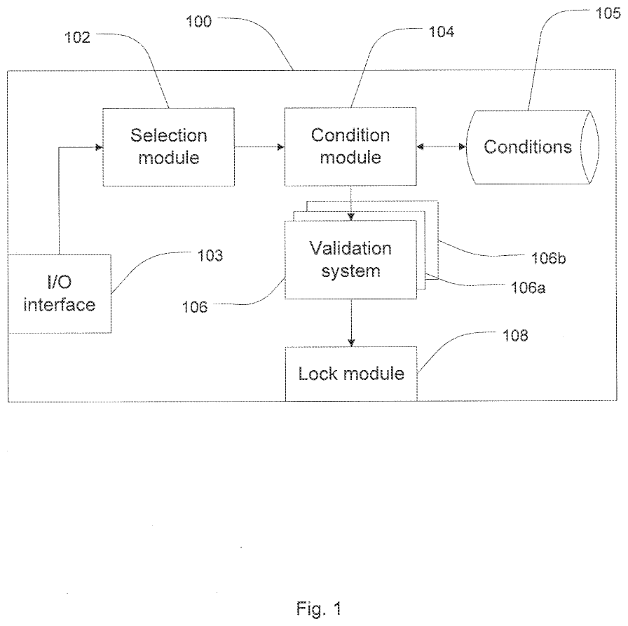

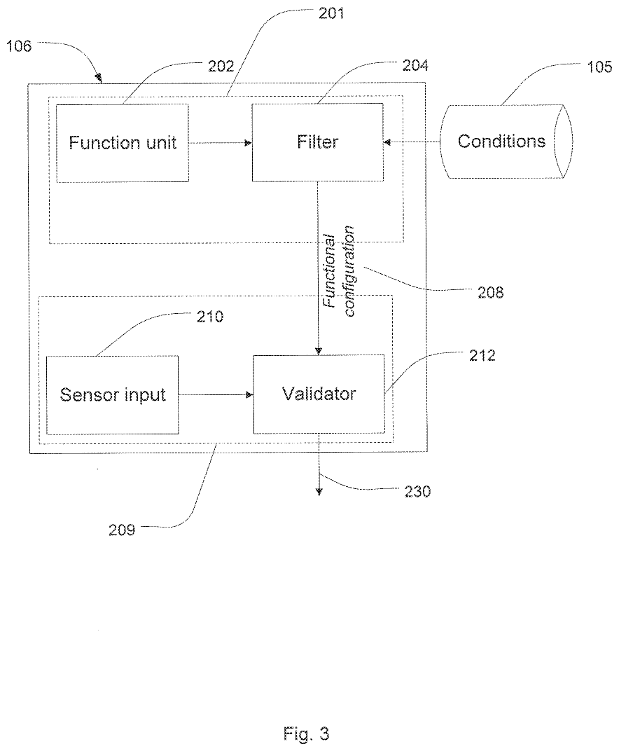

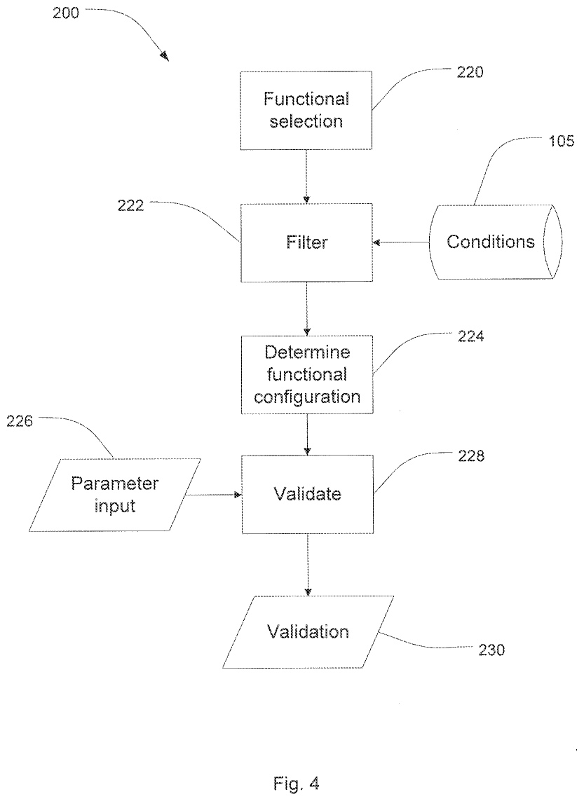

FIG. 1 of the drawings illustrates a multi-functional monitoring system 100. The monitoring system 100 has a selection module 102 configured to receive operational requirements, for example via an I/O interface 103 such as a user interface (e.g. a touchscreen, mouse or keyboard on a computing device). The system 100 also has a condition module 104 in communication with at least one database 105 of conditions. In some embodiments the database 105 forms part of the monitoring system 100. In other embodiments the database 105 is external and separate to the monitoring system 100, but in communication with the monitoring system providing an input to the condition module 104. The condition module 104 selects conditions from the database 105 based on the received operational requirements, and passes these selected conditions to at least one multi-stage validation system 106. The validation system 106 is described in more detail elsewhere herein with reference to FIGS. 3 and 4.

Optionally, the system 100 may include a lock module 108 that locks or unlocks or otherwise controls availability of a context, for example unlocking the context upon successful system validation.

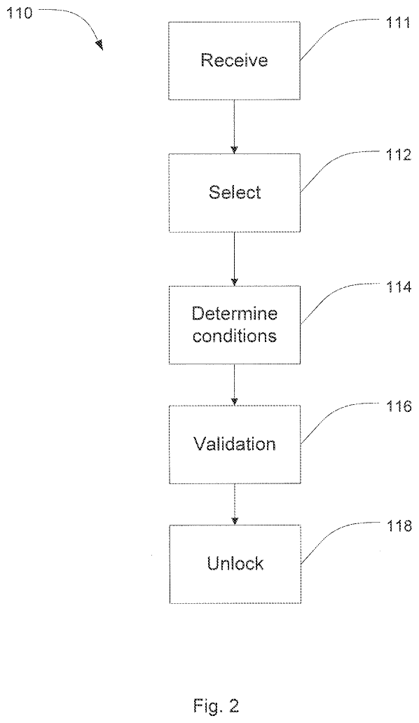

A method of monitoring 110 as implemented by the system illustrated in FIG. 1 may be understood with reference to FIG. 2 of the drawings. At step 111 operational requirements are received.

The operational requirements received may include one or more selected procedure categories and/or one or more selected procedures, one or more item identifiers, and/or a context identifier. As described herein, the operation of a complex system includes executing one or more procedures (for example closing an electronic lock) that each form part of a procedure category (for example securing a premises). Each procedure is executed with respect to one or more identified items (for example a number of doors), and each procedure is executed within an identified context (for example a location, such as the doors of a certain building on the premises). The various operational requirements may each be received from a different source, or they may be received together from the same source (for example selected by a user input, predetermined in association with the complex system, having predefined relationships between e.g. procedures and a related context, etc.)

At step 112 the selection module 102 selects those operational requirements and/or details of the operational requirements to be used in the monitoring and validation processes. At step 114 the condition module 104 queries conditions in the database 105 and selects relevant conditions based on the received and/or selected operational requirements.

The conditions include procedure parameters, which may be predefined and/or configurable parameters required by procedures and/or procedure categories that form part of a complex operation. Procedure parameters may include, for example, required equipment, equipment set up, item requirements for the procedure, item set up or configuration, operator requirements, etc. In the example of securing a premises, equipment may include locks, alarms, movement sensors; equipment set up may include timing settings on locks and alarms, and orientation of sensors; item requirements may be defined as the state of the doors to be locked, e.g. doors must be closed before they are locked (and sensors may be used to determine whether each individual door has been closed or not, before activating a lock); operator requirements may relate to a guard required to lock doors that do not include electronic locks. The conditions may also include item parameters associated with an identified item and/or context parameters associated a selected context.

At step 116 validation of the selected conditions is performed by the validation system 106. At optional step 118 the lock module 108 locks or unlocks the context of the procedure. This may be understood with reference to the above example: where the premises are opened for use, locks are unlocked and alarms disarmed, so that the premises are open for use.

In some embodiments, the system 100 includes at least two multi-stage validation systems 106 (for example systems 106a and 106b as shown in FIG. 1), and the system 100 then determines successful system validation based on a validation 116 from each validation system 106, 106a, 106b etc., or from a defined number of validation systems as required (for example “at least two of the three validation systems must provide a successful validation in order to make a context available and to continue with the procedure”).

In some embodiments each multi-stage validation system 106, 106a, 106b, etc. is associated with one operational function, so that layering the multi-stage validation systems within the monitoring system 100 provides multi-functional operation. Advantageously, this enables efficient cross-functional integration. For example, where a number of doors must be locked in a certain way, and a number of alarms must be set in a certain way, there will be a lock validation system and an alarm validation system, and the monitoring system will ensure that both operational functions are completed before determining that the premises have been secured.

Validation System 106

FIG. 3 of the drawings illustrates an embodiment of a multi-stage validation system 106. This validation system 106 may be an independent system, or may form one or more systems within the monitoring system 100 described with reference to FIGS. 1 and 2.

In a stage 201 of the multi-stage validation system 106 there is a function unit 202 configured to provide a functional selection, and a filter 204 that filters selected conditions as provided by the database 105 of conditions. The filter 204 filters the selected conditions based on the functional selection provided by the function unit 202 in order to provide a functional configuration to a further stage 209 of the multi-stage validation system 106. The selected conditions are a subset of the conditions in the database 105 of conditions, the subset based on at least one operational requirement.

In this further stage 209 the multi-stage validation system 106 has a sensor input 210 that provides at least one parameter to the validator 212 for validation. The validator 212 validates the at least one parameter from the sensor input 210 against the functional configuration 208, in order to provide a validation 230.

A method of monitoring 200 as implemented by the validation system 106 illustrated in FIG. 3 may be understood with reference to FIG. 4 of the drawings.

At 220 a functional selection is made, and at 222 selected conditions provided by the database 105 of conditions are filtered. The filtering 222 is based on the functional selection 220 in order to determine 224 the functional configuration. At least one received parameter input 226 is compared to the functional configuration 224 in order to validate 228 the parameter input 226, thereby providing a validation 230.

### Exemplary Embodiment

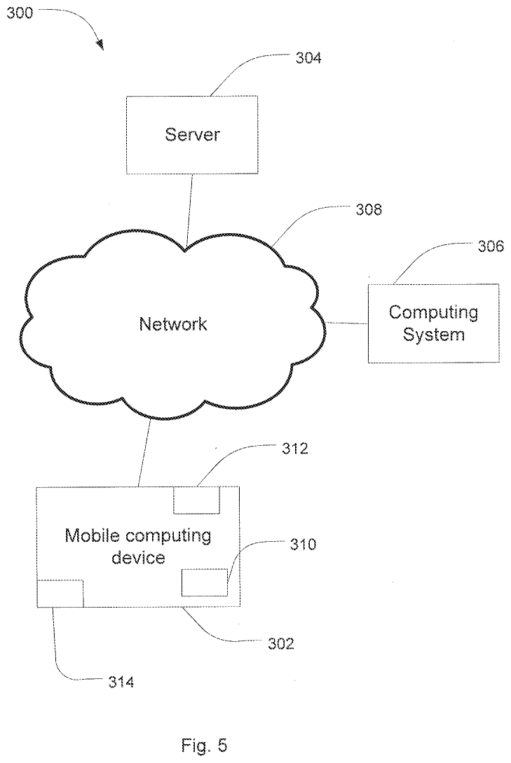

The monitoring system 100 and parts thereof may be implemented using one or more computing devices in any suitable configuration. In one exemplary embodiment illustrated in FIG. 5 of the drawings, a monitoring system 300 includes at least one mobile computing device 302, at least one server 304, and at least one computing system 306, all capable of communicating with one another for example via a network 308, such as the Internet and/or a Wi-Fi network. The mobile computing device 302 may comprise one or more computing devices configured to be communicatively connected to the server 304 and the computing system 306, such as a smart phone, laptop, and/or tablet.

The mobile computing device 302 may run an application 310 residing thereon for allowing a user to enter login information for accessing the server 304 via the network 308. Other computing devices (not shown in FIG. 5) may also have the same or a similar application residing thereon for implementing functionality in accordance with the present disclosure. The server 304 may provide a website or similar platform for implementing functionality described herein. The mobile computing device 302 may include a display controlled to present an interface such that a user can interact with the website or other platform.

The software to create and implement the application may be developed using an integrated development environment, IDE (e.g. one or more of Microsoft Visual Studio, Xamarin Studio, and Android Studio), a coding language (e.g. one or more of C#, Angular JS, Javascript, Objective C, and ASP.NET API), a database (e.g. one or more of SQL Server Azure, and SQLlite), and a User Interface UI Screen Design tool (e.g. one or more of Xamarin iOS, Xamarin Android).

In some embodiments the characteristics of the interface on the display may be configurable depending on, e.g. the type of device used (e.g. a larger tablet or a smaller mobile phone), where the device is used (e.g. outside and independent of the operating room, or specifically in the operating room during or directly before a procedure), who is using the device and how they are able to interact with the user interface depending on their role and the circumstances. Where the computing system 306 is a desktop computer, for example, the user interface to the system may be provided in the form of a web portal with more detail and functionality, requiring more scrolling and clicks to navigate and access or enter data. For a mobile computing device 302, in particular where it is used in an operating room (for example a tablet available in the operating room) the interface is configured to require less screens to click through in order to access data such as equipment and patient positioning. In this way a quick and easy interface is provided, requiring for example only two clicks, as opposed to requiring the user to navigate through three or more windows.

The application 310 of the mobile computing device 302 may be implemented by hardware, software, firmware, or combinations thereof. For example, the application 310 may include one or more processors and memory. The mobile computing device 302 may also include a network interface 312 for communicating with the server 304 and/or other computing devices via the network 308 or another network. Further, the mobile computing device 302 may include a user interface 314. The user interface 314 may include a touch-screen display and/or other I/O components for interfacing with a user. The mobile computing device 302 may include or be in communication with one or more sensor systems configured to provide one or more sensor inputs to the mobile computing device 302. In some embodiments the touch-screen display and/or a mobile device microphone may function as a sensor input to the system whereby configuration information is input.

The operation and features of the multi-functional monitoring system may be understood with reference to a fleet management system, the fleet including various vehicles (items) that are used during various procedures (park, drive, maintenance, etc.)

Once the validation system has been activated for a procedure, the selection module of the validation system receives operational requirements, for example vehicle number one is to park in parking garage number two (i.e. item, procedure, context). The condition module selects conditions from the database based on the received operational requirements, for example the vehicle must arrive at garage number two, the vehicle must be stationary in front of the garage, the vehicle must have a driver, the garage door must be open, the garage light must be on, etc. These are the conditions that must be met for the vehicle to be parked in the specified garage.

In this example there are two multi-stage validation systems that each receives these selected conditions. One system is associated with the vehicle and therefore receives a vehicle-related functional selection. This system filters the conditions to provide a functional configuration relevant to the operation of the vehicle, i.e. where the vehicle needs to be, the state of the vehicle, the presence of a driver (or not, if the vehicle is autonomous).

One or more sensors and/or data inputs associated with the vehicle provides configuration data relating to the vehicle, such as GPS data providing the location and speed of the vehicle. The system validator compares the sensor inputs to the requirements defined by the functional configuration in order to assess whether the requirements are met or not, thereby providing a validation from the vehicle system to the monitoring system.

The second validation system is associated with the parking garages and therefore receives a garage functional selection. The conditions are filtered to provide a functional configuration relevant to the operation of the garages, e.g. selection of which garage or carport, when/how the door is to open/close, etc. One or more sensors and/or data inputs associated with the garages provide relevant configuration data, e.g. proximity sensors that sense the presence of a vehicle, a sensor or manual input to indicate the state of the door, an automated light switch that provides the configuration of the light. The system validator validates the configuration of the garage against the required functional configuration, and provides the monitoring system with a garage-related validation.

Upon receipt of the validations from both systems, the monitoring system is able to determine successful system validation based on the validation from each of the validation systems. If validation is successful, a lock module unlocks the garage entry (for example by displaying a green light, and/or by disengaging one-way access spikes). If validation is unsuccessful, the lock module may lock or maintain a locked indication and/or state of the garage entry.

Once the relevant procedure has been completed, the validation system may reset pending a subsequent activation for another procedure.

### Exemplary Embodiment

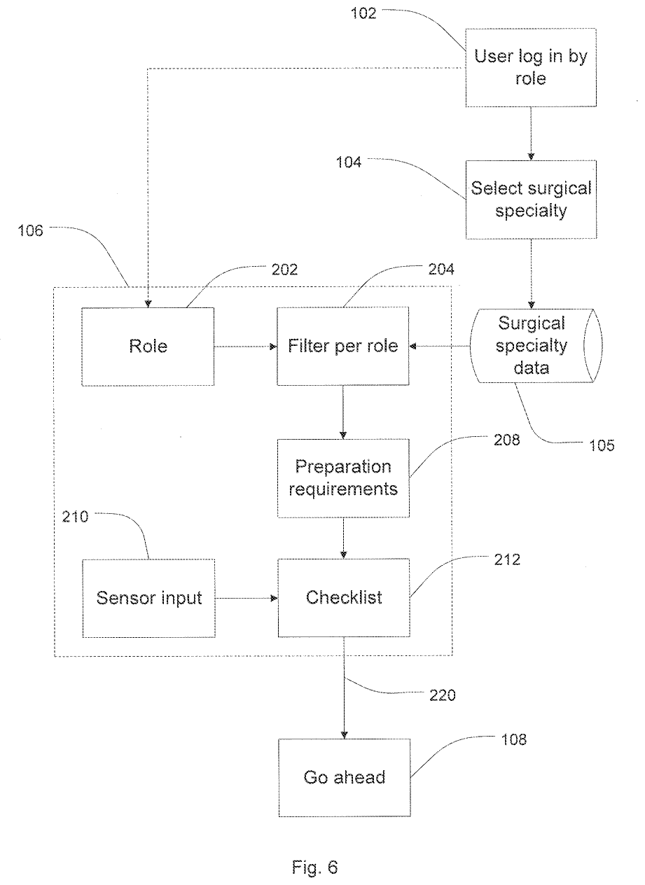

In another example, a monitoring system is used to facilitate cross-functional integration of a perioperative system. Referring to FIG. 6 of the drawings, users access the multi-stage validation systems 106, e.g. via an app on a mobile phone, in order to activate a functional selection according to their individual role (e.g. a nurse, surgeon, technician, orderly, anaesthetist, etc.) Accordingly, the mobile app provides a selection module 102 where users log in and select their role(s). For example, roles for a perioperative system may include operating room nurse, surgical technologist, operating department practitioner, scrub technician, circulating nurse, circulator, scrub nurse, licensed registered nurse, anaesthetic nurse, anaesthetic technician, surgeon, medical doctor, surgical assistant, radiologist, surgical specialist, trainee, nurse, surgical technologist, operating department practitioner, equipment vendor or other third party, sterilisation technicians, etc.

The mobile app provides the user with access to the condition module 104, and the user selects one or more procedure categories in the form of surgical specialties for the relevant procedure, for example dental, foot and ankle, head and neck, organ transplant, robotic, spinal, urology, vascular, trauma, neuro, hand, paediatric, facial maxillary, field surgery, anaesthetics, endoscopy, etc. The selection may be made from a list provided by the medical facility, from a third party database, and/or from a list available directly on the app. In some embodiments the list of surgical specialties may be configurable, e.g. by the medical facility and/or by the user.

In some embodiments the mobile app allows the user to provide further operational requirements such as the specific medical procedure(s) (i.e. procedure identifier), the patient details (i.e. item identifier), and operating room details (i.e. context identifier). In other embodiments one or more of the operational requirements may be provided by the server 304 and/or the computing system 306. For example, the user may be associated with or have access to a specific procedure as scheduled at a particular hospital, so that the hospital computing system 306 provides the details of the procedure, scheduled operating room, and the relevant patient once the user has logged in securely.

In some embodiments, data relating to the monitoring and the validation may be collated, processed, and/or saved for future reference in a process log. In this exemplary embodiment, an example of a process log is where a user, e.g. a nurse, is able to save data in a surgical log based on the name of the procedure. This enables the user to build a professional development log for personal use and/or to provide to professional boards for registration and education, or for any other use of some or all of the data relating to the procedure, such as the use of equipment, the functional configuration (i.e. the Specifications as described below), etc. In some embodiments, the information obtained and saved with respect to exposure and experience within a surgical specialty and/or surgical procedure can also be used to facilitate staff rostering and management of staff (e.g. nurses, surgical technologists and operating department practitioners) based on their experience.

Conditions include procedure parameters associated with the patient, the operating room and/or the procedure. Conditions are typically maintained by one or more databases 105, and these databases 105 may be associated with one or more servers 304 and/or one or more computing systems 306. For example, the hospital computing system 306 may provide details regarding the setup of the particular operating room as well as details regarding the particular patient, while the supporting server 304 may provide generic details regarding the requirements for a procedure category and/or the selected procedure.

The procedure parameters provided by the databases 105 are filtered by the filter 204 of the multi-stage validation system106, which in practice may be implemented using a data filter centralised on the application 310, or distributed amongst the server 304, computing system 306, and/or the mobile computing device 302. The procedure parameters are filtered according to the functional role of each user in order to provide a functional configuration which outlines the perioperative preparation required for each role, e.g. nurse, technician, etc.

In one exemplary embodiment, the functional configuration includes a Preparation Specification, an Operating Room Specification, a Patient Positioning Specification and an Equipment Specification. In some embodiments one or more of the Specifications forming part of the functional configuration may be uploaded and saved to a centralised database, for example a conditions database 105, and/or memory or a database associated with the server 304 and/or the computing system 306.

The Preparation Specification is streamlined with data according to hospital protocols and policies. A checklist based on the Preparation Specification may include how to drape a patient, where to stand and place trolleys, preparation of the patient whilst in the operating room, etc. Data can be shared, synchronised and updated between members of a surgical team and in some embodiments also according to a surgeon's preference. In some embodiments, images and steps can be edited by individuals or locked to groups via one or more mobile applications.

The Operating Room Specification includes details on how to set up and prepare the operating room specific to a surgical procedure and the assigned surgeon. A checklist based on the Operating Room Specification includes equipment checked within the operating room including anaesthetic machine, surgical lights, anaesthetic gases, tool air, tourniquet, etc. The checked equipment may include a sign off (e.g. completed daily), the sign off checking that all safety measures have been met and have been adhered to. The Operating Room Specification may include images and/or the mobile app may include functionality for adding images relating to features in the checklist.

The Patient Positioning Specification provides relevant data required for the selected surgical procedure in view of the specified patient. One or more positioning devices may be specified in the Patient Positioning Specification. Details on time of procedure and length in a specific position are also included, for example lithotomy position, supine, prone, etc. The checklist may include information regarding pressure points to be observed within the type of position. Information may also be provided regarding relevant hospital safety policies. Patient positioning data may be shared amongst team members associated with the specific procedure, and made available to team members that are logged in to the validation system.

The Equipment Specification includes the number of and type of various equipment, such as disposables, drapes, dressings, equipment, extra instruments, surgical gloves, hollow-ware, instrument tray, prep solution, prosthesis, surgical solutions, sutures/tapes/ties, etc. In some embodiments the validation system 106 may be in communication with an inventory system (e.g. a radio-frequency identification (RFID) based system) so that the Equipment Specification may include information regarding stock, location, cost, substitutions, etc. Accordingly, relevant information may be shared, for example, with store managers and/or automatic reordering systems.

In some embodiments surgical instruments listed include details about the equipment, what it is used for, the history and name of the equipment. In some embodiments, if the user does not know what the equipment is called or how it is used, they can take a picture and data points formulated within the software will identify the instrument and how it works. Artificial intelligence in the form of image recognition is used to identify a piece of equipment which has been captured in a photo image.

The Equipment Specification may include equipment resources in the form of one or more of the following: additional information about the equipment (e.g. a video on how to put the equipment together), links to third party websites, links to an information portal about specific surgical equipment, shared data from other team members using the validation system, links to specific implants and prostheses ordered for a procedure, ability to track loan sets and view all loan set equipment, etc. In some embodiments, third party equipment providers are able to provide product information to users who have selected a relevant surgical specialty, for example cardiothoracic providers may provide information relating to heart valves, stents, sutures, grafts etc. if cardiothoracic surgery has been selected.

In some embodiments the monitoring and/or validation systems are in communication with a third party system, the third party being a supplier of equipment, e.g. surgical equipment, monitoring equipment, prosthetics, etc. Conventionally, support is provided by a representative of the third party who is present to provide support to hospital staff. For example, when a new product is used for a hip or knee replacement, the vendor's representative is present and uses a laser pointer to direct where and how to put jigs, saws, etc. together. This can be very stressful for hospital staff learning on the job. The system described herein provides interoperability with third party systems in order to provide guidance with the aid of training material in the form of images, videos, and guidelines of how to put equipment together and how to use equipment.

The functional configuration is selected based on the operational requirements and filtered based on the functional selection and includes various Specifications. One or more of these Specifications may include a training interface to an education and training module. Because the training interface is incorporated in the Specification, this provides easy access to training material from the same user interface (i.e. the same screen) that the user uses to enter or view parameters associated with that Specification of the functional configuration. The education and training module provides images, videos and steps required to carry out a task. A clear theoretical understanding can easily be obtained, in a logical approach, focusing on a procedure, equipment, anaesthetic, positioning, etc. and the implications for each. In this way, a simple user-friendly interface is provided with “one-click” access to training material via the relevant Specification.

Advantageously the education and training module and its training interface enables functionality in the validation systems that enables continuous learning through the various theoretical approaches to learning, such as observation (images and videos), instruction (steps, notes and reflecting on learning outcomes, as shared by experienced staff), and also provides record keeping for procedures and learning experiences. In some embodiments, the validation system may include or be in communication with a learning portfolio that logs details when the user accesses the education and training module and/or equipment resources, for example in a format for the user to save and send to a registration board such as the Australian Health Practitioner Regulation Agency (AHPRA). The system therefore supports “on the job” continued learning and reflection, a requirement of some registration boards.

In some embodiments one or more of the abovementioned Specifications may be user configurable. In some embodiments configurability of the Specifications may be dependent on the function or role type. For example, a surgeon may be able to configure and change any one or more of the Specifications, while other functions or roles (e.g. orderlies) may only be able to configure some, or not be able to configure or amend any of the Specifications. In some embodiments any such changes may be uploaded and saved to a centralised database, for example a conditions database 105, and/or memory or a database associated with the server 304 and/or the computing system 306.

In some embodiments, the validation system may be in communication with an overall hospital surgical schedule. Accordingly, data on the number of surgical instruments and equipment available is tracked and recorded and shared throughout the perioperative suite. Surgical teams can then identify if equipment needs to be fast tracked (e.g. quickly cleaned and resterilised approximately within a specific turnaround time). As a result teams can arrange operating room lists for the day according to the availability of equipment and other resources.

The Equipment Specification may include information relating to pathology (tissue collection) and blood samples. On collection of a pathology form and container, data about specific details on the pathology such as a micro-swab, fresh specimen, frozen specimen, and/or formalin may be uploaded to the monitoring system 100. Such pathology checklists may include information relating to the specific pathology department, details on how much formalin to put with a specimen, container size, how long to keep a fresh specimen, where the tissue collection is, when collection is due, etc.

Similarly, when implants are used, the Equipment Specification may include data on various types of implants available for specific surgical procedures, availability, location, and supporting equipment such as shoulder slings, knee supports, etc.

In some embodiments, the user enters actual configuration data via the application 310, for example via a keyboard or touchscreen in the form of checks in a checklist of tasks completed.

In some embodiments one or more aspects of the configuration (i.e. checklist items) may be input to the validation system via a sensor system. In one example, the placement of equipment may be sensed using an RFID system that tracks the location of equipment, and the RFID system may provide the relevant configuration for the validation assessment.

The configuration (with user-entered and/or sensed automatically entered data) is validated against the functional configuration for the associated role in order to provide a validation for that role in the perioperative preparation. In this way a nurse is able to complete a tailored checklist in preparation for a surgical procedure, and similarly an orderly, surgeon, anaesthetist, etc. is each able to complete a tailored checklist.

Interoperability of Validation Systems, Functional Configurations, and Specifications

The monitoring system determines successful system validation based on a validation from at least one validation system. In one example the combined checklist for perioperative preparation is prepared based on a combination of the various checklists that are based on Specifications for each role associated with the procedure. In some embodiments validation of some of the functional configurations may be a strict requirement, while others may be optional. For example, the combined checklist for perioperative preparation may be prepared based on a combination of a subset of the roles, e.g. a nurse and an anaesthetist checklist. In other embodiments, all the relevant checklists must be completed to attain successful validation.

In some embodiments there is no interoperability of checklists, and only individual checklists are assessed without consideration of other checklists associated with the specific procedure.

In some embodiments the exemplary validation system also includes a lock module in the form of an indicator that allows the procedure to continue in the operating room subject to the satisfactory completion of the relevant checklists. This may be in the form of a “green light”, for example an indication on the display of the mobile app that all checklists have been successfully completed. In other embodiments, this may be implemented by physically unlocking one or more features in the operating room so that the scheduled procedure may continue.

In some embodiments, a reference to an incomplete checklist associated with a procedure (thus resulting in, for example, a context remaining locked) may be shared amongst users associated with the specific procedure. In some embodiments it may then be possible for an alternative function to enter a parameter for a condition of a functional configuration in order to enable validation so as to unlock a context. This is an example of the benefits facilitated by the interoperability of various users' Specifications, so that operating room setup can be performed by a team of professionals working together using the system and with access to information relating to the preparation performed by the various team members.

Referring again to FIG. 5 of the drawings, for this perioperative exemplary embodiment, the backend of the monitoring system 100 may be supported by one or more servers 304, and may be managed for example via an administrator website. The backend may support one or more of the following functionality: a surgical procedure database, customised surgical profiles with equipment locator, complete surgical checklist with operating room supplies, setup and hardware, precise procurement and revenue costings, standardising surgical steps and setups, roster and diary synchronisation, surgical scheduling, linking nurse portfolios to registration boards, recording nurse education portfolios, links to standards of practice (e.g. Australian College of Perioperative Nurses, ACORN), access to hospital policies and procedures, instrument tracking and RFID locator, linking with outside medical or surgical companies for tracking of loan sets and implants, streaming news RSS feeds (e.g. podcasts), streaming training material, communication between surgeons, nurses, reps, orderlies, sterilization department, and/or coordinator, and big data collation etc.

In some embodiments, the procedures and equipment supported by the monitoring and validation systems may be linked to the schedules of medical benefit schemes thereby providing transparency and ease of access to the medical practitioners with respect to the benefits associated with procedures and equipment, thereby supporting decision making and access to information. In embodiments where the conditions provided and parameters required for Specifications of a functional configuration are linked to the requirements of health insurance companies/organisations and/or government bodies such as Medicare in Australia or the NHS in the UK, the resulting checklists help to ensure that procedures are standardised and streamlined. This ensures the safe practices are being met from a healthcare perspective and that auditor recommendations for safe practices are met.

The computing system 306 may provide organisation-level functionality, for example information, support and services provided by hospitals (e.g. private/public, day surgeries, dental suites, etc.), training institutions (e.g. universities, colleges, etc.), and/or medical locum businesses (e.g. nursing agencies, surgical assistant agencies, etc.).

The mobile computing devices 302 may provide functionality via the mobile app for use by a number of different types of users, for example primary medical practitioners (e.g. surgeon, anaesthetist, etc.), supporting medical staff (e.g. nurses, surgical technologists, etc.), and/or third party or auxiliary staff (e.g. nursing students, sterilisation department, etc.).

Advantageously, the multi-functional monitoring system that incorporates multi-stage validation systems, and the related methods as described herein, enable complex operations to be divided into sub-systems according to different functions, and roles, making them more manageable. By defining requirements for the sub-systems based on functional requirements, large complex operations may be divided into smaller, simpler tasks that can be managed more efficiently, and can be automated and tracked. In this way, the tasks for functional sub-systems are more clearly defined, making it possible to demarcate handovers in order to more efficiently execute tasks and assign responsibility. Advantageously, the interoperability of the validation systems associated with various functions of the multi-functional system makes for a more efficient validation and monitoring solution that is able to incorporate inputs associated with more than one function for any given procedure. This enables efficient cross-functional integration.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A perioperative monitoring system for medical personnel in a medical environment, the system comprising:
a selection module configured to receive operational requirements, the operational requirements including a procedure identifier, an item identifier, and a medical environment identifier;
a condition module responsive to the selection module and in communication with a database of conditions, the conditions including one or more checklists selected from the group consisting of a preparation specification, an operating room specification, a patient position specification, and an equipment specification, wherein the condition module is configured to select conditions from the database based on the received operational requirements;
at least one validation system that receives the selected conditions from the condition module, wherein the at least one validation system includes a filter that filters the selected conditions based on selected ones of the medical personnel in order to provide a functional medical configuration to be implemented by the selected medical personnel, the at least one validation system configured to determine a validation based on a successful implementation of the functional medical configuration by the selected medical personnel; and
a lock module communicatively connected to the at least one validation system and the medical environment, the lock module being configured to lock the medical environment and to unlock the medical environment for the selected medical personnel upon validation being determined by the at least one validation system.

2. The monitoring system of claim 1, which includes two or more validation systems each associated with a respective functional medical configuration and respective medical personnel, and wherein the validation is determined based on a successful implementation of each respective functional medical configuration by respective selected medical personnel.

3. The monitoring system of claim 2, wherein the respective functional medical configurations complement one another so as to be interoperable.

4. The monitoring system of claim 1, wherein the one or more checklists are selected based on the procedure identifier.

5. The monitoring system of claim 1, wherein the one or more checklists are user-configurable.

6. The monitoring system of claim 1, further including:
a computing system or device having a display; and
a user interface provided on the display, the user interface being functionally configurable based on at least one of the computing system or device type, the computing system or device location, and the selected medical personnel.

7. The monitoring system of claim 1, wherein the at least one validator system includes a sensor input that provides at least one parameter for determining the validation.

8. The monitoring system of claim 1, wherein the procedure identifier is received from the database of conditions or from medical personnel input.

9. The monitoring system of claim 1, wherein the procedure identifier is configurable.
